(19) United States
(12) Patent Application Publication
(10) Pub. No.: US 2024/0018692 A1
(43) Pub. Date: Jan. 18, 2024

(54) DEVICE AND METHOD FOR PRODUCING AN ANISOTROPIC FIBRE STRUCTURE BY ELECTROSPINNING

(71) Applicants: MEDIZINISCHE UNIVERSITÄT WIEN, Wien (AT); LUDWIG BOLTZMANN GESELLSCHAFT-ÖSTERREICHISCHE VEREINIGUNG ZUR FÖRDERUNG DER WISSENSCHAFTLICHEN, WIEN (AT)

(72) Inventors: Christian GRASL, Wien (AT); Heinrich SCHIMA, Wien (AT); Helga BERGMEISTER, Wien (AT)

(21) Appl. No.: 18/257,278

(22) PCT Filed: Dec. 16, 2021
(86) PCT No.: PCT/EP2021/086182

§ 371 Date: Jun. 13, 2023

(30) Foreign Application Priority Data
Dec. 17, 2020 (EP) 20214821.9

(51) Int. Cl. D01D 5/00; D01F 6/62; D01F 6/70; A61F 2/06

(52) U.S. Cl. CPC D01D 5/0092; D01D 5/003; D01F 6/625; D01F 6/70; A61F 2/06; D10B 2331/041; D10B 2331/10; D10B 2509/06; A61F 2240/001

(57) ABSTRACT

Device and method for producing an anisotropic fibre framework (10), in particular a stent, by electrospinning, comprising a reservoir (1), a spinneret (2) for dispensing a material jet (8), and an electrically conductive depositing spindle (3) which is rotatable relative to the spinneret (2), wherein at least one, preferably variable, two-pole electrical DC voltage source (4, 4a, 4b) is provided for providing an electrical potential difference between the spinneret (2) and the depositing spindle (3), and wherein a, preferably earthed, housing (7) which at least partially surrounds the depositing spindle (3) and the spinneret (2) is provided, wherein a controlled switching unit (5) is provided, this being designed to isolate the depositing spindle (3) from the DC voltage source (4, 4a, 4b) at least once, preferably several times, during the electrospinning.

The invention relates to a device and a method for producing an anisotropic fibre structure by electrospinning.

Electrospinning is a technology for producing fibres from natural and synthetic polymers with diameters of approximately 30 nm to 3 μm. The fibre is formed by the acceleration of the charge carriers induced in the polymer in a strong electric field, which ultimately leads to the stretching of the viscoelastic material. In the most basic case, the electric field is generated by applying a high voltage between a reservoir containing the polymer solution and a separating spindle on which the fibres are deposited.

The reservoir is connected to a spinneret through which the polymer solution jet is ejected. By selecting the spinning parameters (e.g. distance between reservoir and separating spindle, viscosity, surface tension and conductivity of the polymer solution, selected high voltage, . . . ), the properties of the fibres formed (diameter, fibre spacing, homogeneity, . . . ) can be affected.

In particular, the production of medical vascular prostheses requires generating anisotropic, mostly tubular fibre structures (so-called grafts or stent grafts). To produce tubular fibre structures by electrospinning, the use of a rotatable separating spindle onto which the material jet is deposited is known. The separating spindle is elongated and rod-shaped and is also called a mandrel.

To provide an electrical potential difference between the spinneret and the separating spindle, the spinneret is connected to the first electrical pole of a two-pole electrical DC voltage source. In particular, this may be a variably adjustable high voltage generator. A positive or negative high voltage may be applied to the polymer solution, creating a potential difference between the reservoir or spinneret and the separating spindle.

It has been shown that the biocompatibility of the produced grafts and in particular the ingrowth of endogenous cells into the grafts is dependent on the local structuring of the produced fibre structure. Consequently, one of the objects of the invention is to provide a method which is as simple and robust as possible, as well as a corresponding device, which enables local structuring of the produced fibre structure.

According to the invention, this object is achieved by a device according to claim 1 and a method according to claim 8.

A device according to the invention is configured for producing an anisotropic fibre structure, in particular a vascular prosthesis, by electrospinning and comprises a reservoir, a spinneret for ejecting a material jet and an electrically conductive separating spindle which is rotatable relative to the spinneret. At least one electrical DC voltage source is provided to supply an electrical potential difference between the spinneret and the separating spindle. In particular, it may be a variable two-pole high-voltage source.

Furthermore, a housing is provided which at least partially encloses the separating spindle and the spinneret, the housing preferably being grounded. A controlled switching unit is provided which is configured to alternately connect the separating spindle to the DC voltage source during electrospinning, i.e. to apply it to a defined potential, and to disconnect it from the source again, i.e. to leave the separating spindle floating. The separating spindle may be made of or comprise an electrically conductive material, for example steel, aluminium or copper. The separating spindle may also be referred to as the depositing spindle or depositing electrode.

In particular, two embodiments may be provided according to the invention:

In a first embodiment, the spinneret is connected to a first electrical pole (e.g. the positive pole) of the DC voltage source and the separating spindle is connected to a second electrical pole (e.g. the negative pole) of the DC voltage source. The switching unit is configured to connect the second electrical pole of the DC voltage source to the separating spindle at least once, preferably several times, during electrospinning and to disconnect it from the separating spindle again after a certain time period. In particular, the separating spindle is thereby switched between the ground potential (0 V) and an undefined potential (floating). This embodiment may be provided when the potential difference that is generatable by the DC voltage source (e.g. 15 kV) is sufficient for the spinning process; during the spinning process, the spinneret is at +15 kV and the separating spindle is at 0 V or floating.

In a second embodiment, two DC voltage sources are provided, wherein the spinneret is connected to a first electrical pole (e.g. the positive pole) of the first DC voltage source, and the separating spindle is connected to a first electrical pole (e.g. the positive pole) of the second DC voltage source. The switching unit is configured to connect the first electrical pole of the second DC voltage source to the separating spindle at least once, preferably several times, during electrospinning and to disconnect it from the separating spindle again after a certain time period.

This second embodiment may be provided in particular if the potential difference that is generatable by the DC voltage source (e.g. 15 kV) is not sufficient for the spinning process; two DC voltage sources are then used. During the spinning process, the spinneret is at +15 kV and the separating spindle is at a potential value of below 0 V, for example −2 kV, or floating, so that a potential difference of 17 kV is generated. In this case, the second electrical poles of the DC voltage sources (the negative poles) are electrically connected.

By switching between 0 V (or a negative potential) according to the invention and an undefined (floating) potential during electrospinning, it is achieved that the structural properties of the produced fibre structure change during the spinning process. This allows the thickness, texture and mechanical properties of the fibre structure produced to be changed in a simple way, purely electrically and without mechanical intervention.

For example, it has been shown that some materials tend to form waves or flaps during electrospinning when the separating spindle is at GND potential, i.e. the outer circumference of the fibre structure becomes irregular and the fibre structure extends inhomogeneously outwards in the radial direction. By switching the second electrical pole of the DC voltage source after a certain spinning time according to the invention, it has surprisingly been shown that this suppresses the formation of waves or flaps on the outer circumference, as they contact the original cylindrical fibre structure, forming cavities in the fibre structure. With suitably selected time periods, cavities with a typical diameter of more than 5 μm are formed.

By selecting the amount of time between switching, the size of the cavities formed by the flaps in the fibre structure may be varied. In particular, it has been shown that the longer the second pole is connected to the separating spindle, the larger the flaps and thus also the cavities.

In other words, if the separating spindle is at a defined potential (in particular at GND or a potential lower than 0 V), then irregularities form in the fibre structure, which are closed when the separating spindle is disconnected from the defined potential and floats. This closing of the irregularities thus creates cavities whose expansion is affectable by the switching of the voltage ratios.

Since the waves or flaps formed have different macroscopic properties than the basic structure, for example different strength and elasticity, intermediate layers with the desired properties may be created in the fibre structure in a targeted manner.

Both the cavities produced in this way and the intermediate layers in the fibre structure offer advantages when using the fibre structures formed as vascular prostheses, for example with regard to their biocompatibility or adaptation to the flexibility of the treated vessels.

According to the invention, a return electrode may be provided behind the separating spindle with respect to the ejected material jet. Such a return electrode may serve to stabilise the material jet when the separating spindle is at an undefined potential, i.e. floating. For this purpose, the switching unit is configured to connect the second electrical pole of the DC voltage source, or the first electrical pole of the second DC voltage source, to the return electrode during electrospinning when the separating spindle is floating.

According to the invention, a housing is provided which at least partially encloses the separating spindle and the spinneret. The housing may be connected to the second electrical pole (negative pole or GND) of the DC voltage source or to the second electrical poles (negative poles or GND) of the DC voltage sources. In particular, the housing may be grounded. This ensures that the housing of the device is grounded, i.e. at a potential of 0 V, thus forming a “Faraday cage”.

The potential difference thus generated may be about 5 kV to 20 kV, preferably 8 kV to 17 kV, particularly preferably 10 kV to 15 kV, whereby the potential at the separating spindle is preferably smaller than the potential at the spinneret, in particular smaller than or equal to 0 V, for example −2 kV.

The switching of the DC voltage source(s) may be performed manually or by an electronic control unit. Such an electronic control unit may be provided in particular for controlling at least the DC voltage source(s) and the switching unit. However, the control unit may also control other elements of the device according to the invention, for example a valve of the reservoir to control the ejection of the material, or motors to rotate and/or displace the separating spindle.

The separating spindle may be configured as a spindle which is rotatable relative to the spinneret and which is rotatable in an axis extending substantially orthogonally to the ejected material jet and/or is displaceable along this axis. In particular, it may be provided that the separating spindle is reciprocally movable along this axis in a sinusoidal or triangular oscillation in order to produce an elongated fibre structure.

A sinusoidal oscillation of the displacement results in widened end regions of the fibre structure, which may subsequently serve as support rings of the vascular prosthesis. Alternatively or additionally, it may be provided that the return electrode is reciprocally movable along this axis in a sinusoidal or triangular oscillation in order to produce the elongated extension of the fibre structure. Furthermore, by slowing or reducing the oscillation of the displacement at predetermined positions, a local thickening of the vessel wall thickness may be achieved in order to prevent collapse or external compression of the vessel in the patient through to the resulting support rings. The separating spindle may also be displaceable in the direction of the material jet in order to be able to adjust the distance between the spinneret and the separating spindle.

The separating spindle may have a diameter of about 2 mm and a rotational speed during electrospinning of about 250 revolutions per minute. The distance between the spinneret and the separating spindle may be about 5 cm to 12 cm, for example about 8 cm. The diameter of the separating spindle may also be considerably larger, for example 5 mm, 10 mm, 20 mm, 30 mm or 36 mm, for example in case the vascular prosthesis is to be inserted into the aorta of an adult. In particular, a diameter of the separating spindle of about 2 mm may be provided for the creation of vascular prostheses for the replacement of smaller vessels (arteries, but also veins, lymphatic vessels or cerebrospinal fluid drainage systems). Alternatively, a diameter of about 4 mm to about 8 mm may be provided for medium vessels and dialysis shunts, but larger diameters may be provided to the point of replacing sections of the aorta, pulmonary artery or large veins.

If a larger diameter of the separating spindle is selected, the rotational speed is preferably also reduced accordingly in order to achieve a substantially equivalent peripheral speed. For example, with a diameter of the separating spindle of about 5 mm, a rotational speed of about 80-120 revolutions per minute may be provided. For large diameters, several spinnerets may also be provided.

In the area between the spinneret and the separating spindle, stabilising electrodes may further be provided, which are preferably plate-shaped and are preferably connected to the first electrical pole or to the second electrical pole of the DC voltage source(s). Such stabilising electrodes may be used to stabilise and, optionally, to focus the ejected material jet.

The ejected material may be a mixture of a polymer and a solvent. For example, the material jet may comprise a solution of 5 wt.-% polydioxanone (PDS) in hexafluoro2propanol or a solution of 5 wt.-% thermoplastic polyurethane (TPU) in hexafluoro2propanol.

The invention further relates to a method of producing an anisotropic fibre structure, in particular a vascular prosthesis by electrospinning, wherein a material jet is guided from a reservoir via a spinneret onto a rotating, electrically conductive separating spindle and wherein an electrical potential difference is formed between the spinneret and the separating spindle by connecting the spinneret and the separating spindle to a preferably variable, two-pole electrical DC voltage source.

According to the invention, a controlled switching unit is provided which connects the separating spindle to the DC voltage source at least once, preferably several times, during the spinning process and disconnects it from the DC voltage source again after a predetermined time period. Through this change in the electrical potential difference between the separating spindle and the spinneret during electrospinning, a local structuring of the deposited fibre structure is obtained.

In a first embodiment, the potential difference is formed by connecting the spinneret to a first electrical pole of the DC voltage source and connecting the separating spindle to a second electrical pole of the DC voltage source. The switching unit connects the second electrical pole (negative pole, GND) of the DC voltage source to the separating spindle and disconnects them again after a determined time period. By changing the electrical potential difference between the separating spindle and the spinneret once or several times during electrospinning, a local structuring of the deposited fibre structure may be obtained.

In an alternative embodiment of the method according to the invention, the potential difference is formed by connecting the spinneret to a first electrical pole of a first DC voltage source and connecting the separating spindle to a first electrical pole of a second DC voltage source.

During electrospinning, the switching unit connects the first electrical pole of the second DC voltage source at least once, preferably several times, to the separating spindle and disconnects it therefrom again after a certain time period.

In both embodiments, it may be provided that the switching unit disconnects the second electrical pole of the DC voltage source, or the first electrical pole of the second DC voltage source, from the separating spindle at least once, preferably several times, and connects it to a return electrode arranged behind the separating spindle with respect to the material jet.

During electrospinning, the separating spindle may rotate relative to the spinneret along an axis extending substantially orthogonally to the ejected material jet and/or be displaced along this axis.

In particular, the separating spindle may be reciprocated along this axis in a sinusoidal or triangular oscillation. Alternatively or additionally, the return electrode may also be reciprocated along this axis in a sinusoidal or triangular oscillation.

During electrospinning, the switching unit may connect the separating spindle to the DC voltage source for a first time period t1 and then disconnect the separating spindle from the DC voltage source for a second time period t2. The time periods t1, t2 may each be about 5 minutes to 10 minutes. The process may be repeated about 5 to about 10 times.

Of course, the process may also be performed in reverse order, wherein the switching unit first disconnects the separating spindle from the DC voltage source for a time period t2 and then connects the separating spindle to the DC voltage source for a time period t1.

The time periods t1 and t2 may also be different. For example, the time period tl may be about 5 to 10 minutes and the time period t2 may be about 2 to 5 minutes.

However, the time period t2 is preferably shorter than the time period t1. The total duration of electrospinning may be the sum of tl and t2. The switching unit may be configured to set these time periods and the switching time differently depending on the application and the spun material, corresponding values being obtained from a database, for example.

The rotational speed of the separating spindle may be about 250 revolutions per minute and the diameter of the separating spindle may be about 2 mm, 5 mm, 10 mm, 20 mm, 30 mm or 36 mm. The delivery rate of the material jet from the reservoir may be about 0.5-2 ml/h, in particular about 0.7 ml/h.

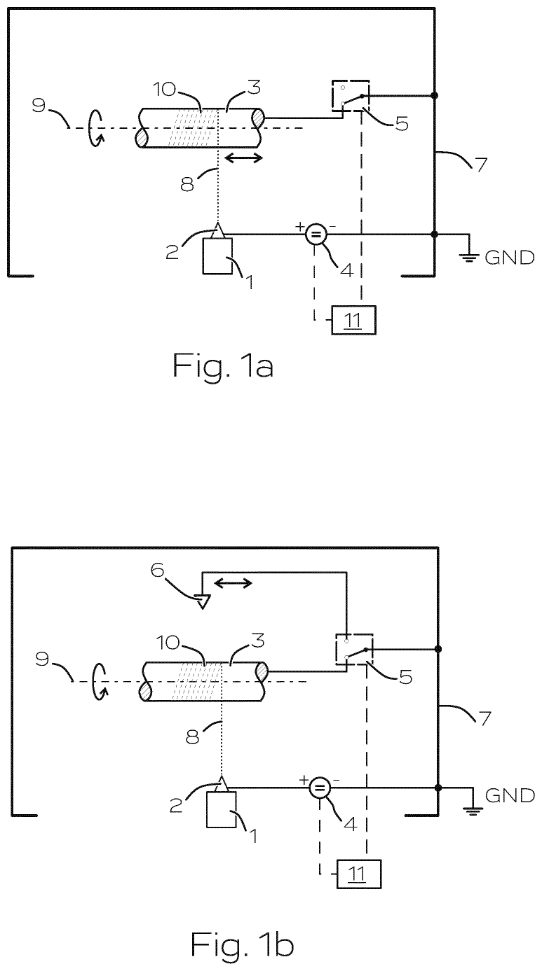

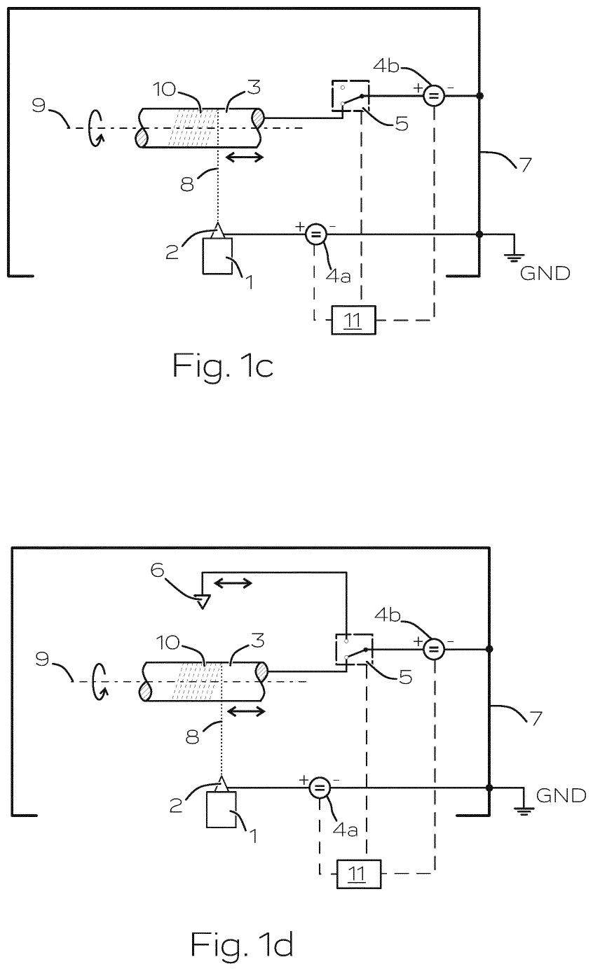

Further features of the invention become apparent from the claims, the following description of the embodiments and the figures. In the figures: FIG. 1a shows a schematic representation of a first embodiment of a device according to the invention; FIG. 1b shows a schematic representation of a second embodiment of a device according to the invention; FIG. 1c shows a schematic representation of a third embodiment of a device according to the invention;

FIG. 1d shows a schematic representation of a fourth embodiment of a device according to the invention;

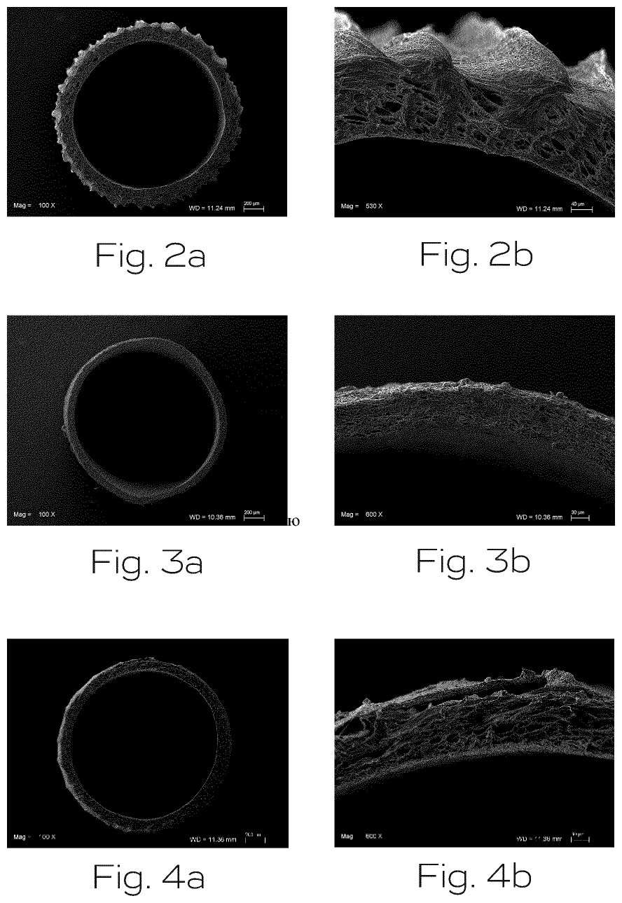

FIGS. 2a - 3b show microscopic images of a cross section of two fibre structures produced using conventional methods;

FIGS. 4a - 4b show microscopic images of a cross section of a fibre structure produced using a method according to the invention.

FIG. 1a shows a schematic representation of a first embodiment of a device according to the invention. The device is configured to produce an anisotropic fibre structure 10 in the form of a vascular prosthesis by electrospinning and comprises a reservoir 1 in which a polymer solution is provided and a spinneret 2 connected thereto for ejecting a material jet. The spinneret 2 is connected to a first pole of an electrical DC voltage source 4, in this case the positive pole of this DC voltage source 4.

The negative pole of the DC voltage source 4 is grounded (GND) and connected to a housing 7. A rotatable separating spindle 3 is provided at a distance of about 8 cm from the spinneret 2. The DC voltage source 4 provides a high voltage of about 8 kV.

The separating spindle 3 is electrically conductive, for example made of steel, and is connected to the housing 7, i.e. the negative pole of the DC voltage source 4, via a switching unit 5. An electrical potential difference is formed between the spinneret 2 and the separating spindle 3, which causes material exiting the spinneret 2 to be dispensed onto the rotating separating spindle 3 in the form of a material jet 8. The rotation of the separating spindle 3 causes the material jet 8 to wind up and form a schematically shown tubular fibre structure 10. By the periodic sinusoidal or triangular displacement of the separating spindle 3 along the axis 9, indicated by a double arrow, the length of the produced fibre structure 10 may be adjusted.

A controlled electronic switching unit 5 is provided, which is designed to connect the second electrical pole of the DC voltage source 4 to the electrically conductive separating spindle 3 during electrospinning and to disconnect it therefrom again. In other words, the separating spindle alternates between a state (a) in which it is held at GND potential and a state (b) in which it freely floats.

In state (b), a material jet 8 is nonetheless formed, since in this case a potential difference is established between the spinneret 2 and the housing 7, so that the material jet 8 is attracted by the housing 7 and comes into contact with the separating spindle 3.

In state (a), a structure with faults on the surface is formed, which were observed as waves or radially outwardly directed flaps running in the longitudinal direction. In state (b), a largely uniform structure without larger cavities is formed. By switching at least once between state (a) and state (b), flap formation only occurs in state (a), while in state (b) the formed flaps fold homogeneously over the already existing fibre structure. This results in the formation of smaller or larger cavities in the peripheral wall of the fibre structure.

FIG. 1b shows a schematic representation of a second embodiment of a device according to the invention. It largely corresponds to the first embodiment with the difference that an electrically conductive return electrode 6 is provided, arranged behind the separating spindle 3 with respect to the ejected material jet 8. The switching unit 5 switches the second electrical pole (the negative pole) of the DC voltage source 4 between the separating spindle 3 and the return electrode 6. In state (b), i.e. when the separating spindle 3 is floating, fibres also form between the return electrode 6 and the separating spindle 3, which are wound up by the spindle due to its rotation, but without forming flaps. By the periodic sinusoidal or triangular displacement of the return electrode 6 parallel to axis 9, the length of the produced fibre structure 10 can be adjusted.

FIG. 1c and FIG. 1d show a schematic representation of a third and fourth embodiment of a device according to the invention. These embodiments correspond to those in FIG. 1a and FIG. 1b, however, two DC voltage sources 4a, 4b are provided. This allows the generation of higher potential differences than with a single DC voltage source.

The spinneret 2 is connected to the first electrical pole of the first DC voltage source 4a and the separating spindle 3 is connected to a first electrical pole of the second DC voltage source 4b. The switching unit 5 is configured to alternately connect and disconnect the first electrical pole of the second DC voltage source 4b to and from the separating spindle 3 during electrospinning.

In the exemplary embodiment according to FIG. 1d, a return electrode 6 arranged behind the separating spindle 3 with respect to the ejected material jet 8 is provided, wherein the switching unit 5 is configured to alternately connect the first electrical pole of the second DC voltage source 4b to the separating spindle 3 and to disconnect it from the separating spindle 3 and connect it to the return electrode 6 during electrospinning. In this exemplary embodiment, both the separating spindle 3 and the return electrode 6 are periodically displaceable in a sinusoidal or triangular manner along the direction indicated by the double arrows, in order to adjust the length of the produced fibre structure 10.

In both exemplary embodiments according to FIG. 1c and FIG. 1d, a housing 7 partially enclosing the separating spindle 3 and the spinneret 2 is provided, the housing being connected to the second electrical poles (the negative poles) of the DC voltage sources 4a, 4b and grounded via a GND connection.

In an exemplary embodiment of the invention not shown, 5 wt.-% thermoplastic polyurethane (TPU, described in S. Baudis et al., Hard-block degradable thermoplastic urethane-elastomers for electrospun vascular prostheses, J. Polym. Sci. Part A Polym. Chem. 50 (2012) 1272-1280) were dissolved in hexafluoroisopropanol and processed into continuous nanofibres using an electrospinning device according to the invention, which were wound into a tube on the rotating separating spindle. The distance between the needle tip of the spinneret and the separating spindle was about 8 cm. The electrospinning device was placed as a whole in a Faraday cage (housing) and operated in a class 1000 clean room at a temperature of 24° C. and 34% RH. The inflow of polymer solution into the spinneret was set to about 0.7 ml/h and a voltage of about 12 kV was applied to the spinneret. The electrospun tube thus obtained on the separating spindle had an inner diameter of 1.5 mm and a wall thickness of about 300 μm.

FIGS. 2a-3b show microscopic images of a cross section in a complete view and in a magnified view of various fibre structures produced using conventional methods.

In the fibre structure according to FIGS. 2a-2b, the separating spindle 3 was kept at GND potential for the entire duration of the spinning process, corresponding to the above defined state (a). This results in a fibre structure with numerous radial waves or flaps running in the longitudinal direction of the tubular fibre structure formed. In the fibre structure according to FIGS. 3a-3b, the separating spindle 3 was kept floating, i.e. without dedicated potential, for the entire duration of the spinning process according to the state (b) defined above, a return electrode being provided which was kept constantly at GND potential during the spinning process. This results in a fibre structure without faults.

FIGS. 4a- 4b show microscopic images of a cross section in a complete view and in a magnified view of various fibre structures produced using a method according to the invention. In the fibre structure according to FIGS. 4a-4b, the separating spindle 3 was kept at GND potential for a time tl during the spinning process and then kept floating for a time t2. The electrospinning process extended over the time period t1+t2. The values of t1 and t2 were about 5 minutes and 15 minutes respectively; the total spinning process about 20 minutes. This results in a fibre structure with numerous cavities in the outer wall running in the longitudinal direction of the tubular fibre structure formed, but without external faults. Such a fibre structure may be used particularly advantageously in vascular surgery, as the cavities promote, among other things, the ingrowth of human tissue. However, the invention is not limited to the illustrated exemplary embodiments, but rather comprises all devices and methods in the context of the following patent claims. In particular, the invention is not limited to devices and methods for producing vascular prostheses.

### List of reference signs

1 Reservoir
2 Spinneret
3 separating spindle
4a, 4b DC voltage source
5 Switching unit
6 Return electrode
7 Housing
8 Material jet
9 Axis
Fibre structure
11 Control unit

1. A device for producing an anisotropic fibre structure (10), in particular a vascular prosthesis, by electrospinning, comprising a reservoir (1), a spinneret (2) for ejecting a material jet (8) and an electrically conductive separating spindle (3) which is rotatable relative to the spinneret (2), wherein at least one preferably variable, two-pole electrical DC voltage source (4, 4a, 4b) for providing an electrical potential difference between the spinneret (2) and the separating spindle (3) is provided, and a preferably grounded housing (7) at least partially enclosing the separating spindle (3) and the spinneret (2) is provided,
characterised in that
a controlled switching unit (5) is provided which is configured to disconnect the separating spindle (3) from the DC voltage source (4, 4a, 4b) at least once, preferably several times, during electrospinning.

2. The device according to claim 1, characterised in that the spinneret (2) is connected to a first electrical pole of the DC voltage source (4) and the separating spindle (3) is connected to a second electrical pole of the DC voltage source (4), wherein the switching unit (5) is designed to disconnect the second electrical pole of the DC voltage source (4) from the separating spindle (3) at least once, preferably several times, during electrospinning.

3. The device according to claim 1, characterised in that two DC voltage sources (4a, 4b) are provided, wherein the spinneret (2) is connected to a first electrical pole of the first DC voltage source (4a) and the separating spindle (3) is connected to a first electrical pole of the second DC voltage source (4b), and wherein the switching unit (5) is designed to disconnect the first electrical pole of the second DC voltage source (4b) from the separating spindle (3) at least once, preferably several times, during electrospinning.

4. The device according to claim 2, characterised in that a return electrode (6) is provided behind the separating spindle (3) with respect to the ejected material jet (8), the switching unit (5) being designed to disconnect
a. the second electrical pole of the DC voltage source (4) or
b. the first electrical pole of the second DC voltage source (4b) from the separating spindle (3) at least once, preferably several times, during electrospinning and to connect it to the return electrode (6).

5. The device according to claim 2, characterised in that the housing (7) is connected
a. to the second electrical pole of the DC voltage source (4) or
b. to the second electrical poles of the DC voltage sources (4a, 4b).

6. The device according to claim 1, characterised in that the potential difference is about 5 kV to 20 kV, preferably 8 kV to 17 kV, particularly preferably 10 kV to 15 kV, wherein the potential at the separating spindle (3) is preferably smaller than the potential at the spinneret (2), in particular smaller than or equal to 0 V, for example −2 kV.

7. The device according to claim 1, characterised in that the separating spindle (3) is rotatable in an axis (9) extending substantially orthogonally to the ejected material jet (8) and/or is displaceable along this axis (9), wherein the separating spindle (3) is reciprocally movable in particular along the axis (9) in a sinusoidal or triangular oscillation.

8. The device according to claim 1, characterised in that the controlled switching unit (5) is configured to alternately connect and disconnect the separating spindle (3) with/from the DC voltage source (4, 4a, 4b) during electrospinning.

9. The device according to one of claims 1 to 8claim 1, characterised in that an electronic control unit (11) is provided for controlling at least the DC voltage source (4, 4a, 4b) and the switching unit (5).

10. A method of producing an anisotropic fibre structure (10), in particular a vascular prosthesis by electrospinning, wherein a material jet (8) is guided from a reservoir (1) via a spinneret (2) onto a rotating, electrically conductive separating spindle (3), wherein an electrical potential difference is formed between the spinneret (2) and the separating spindle (3) by connecting the spinneret (2) and the separating spindle (3) to a preferably variable, two-pole electrical DC voltage source (4, 4a, 4b),
characterised in that
a controlled switching unit (5) disconnects the separating spindle (3) from the DC voltage source (4, 4a, 4b) at least once, preferably several times, during electrospinning, so that local structuring of the deposited fibre structure (10) is achieved by changing the electrical potential difference between the separating spindle (3) and the spinneret (2) during electrospinning.

11. The method according to claim 10, characterised in that the potential difference is formed by connecting the spinneret (2) to a first electrical pole of the DC voltage source (4) and connecting the separating spindle (3) to a second electrical pole of the DC voltage source (4), wherein the switching unit (5) disconnects the second electrical pole of the DC voltage source (4) from the separating spindle (3) at least once, preferably several times, during electrospinning.

12. The method according to claim 11, characterised in that the potential difference is formed by connecting the spinneret (2) to a first electrical pole of a first DC voltage source (4a) and connecting the separating spindle (3) to a first electrical pole of a second DC voltage source (4b), wherein the switching unit (5) disconnects the first electrical pole of the second DC voltage source (4b) from the separating spindle (3) at least once, preferably several times, during electrospinning.

13. The method according to claim 11, characterised in that the switching unit (5) disconnects the second electrical pole of the DC voltage source (4) or the first electrical pole of the second DC voltage source (4b) from the separating spindle (3) at least once, preferably several times, during electrospinning and connects it to a return electrode (6) arranged behind the separating spindle (3) with respect to the material jet (8).

14. The method according to claim 10, characterised in that the separating spindle (3) rotates relative to the spinneret (2) along an axis (9) extending approximately orthogonally to the ejected material jet (8) and/or is displaced along this axis (9) during electrospinning, in particular in that the separating spindle (3) is reciprocated along the axis (9) in a sinusoidal or triangular oscillation.

15. The method according to claim 10, characterised in that the switching unit (5) connects the separating spindle (3) to the DC voltage source (4, 4a, 4b) for a first time period tl during electrospinning, and then disconnects the separating spindle (3) from the DC voltage source (4, 4a, 4b) for a second time period t2, the time periods t1, t2 each being about 5 to 10 minutes and this process being repeated 5 to 10 times to produce the fibre structure (10).

16. The method according to claim 10, characterised in that the delivery rate of the material jet (8) from the reservoir (1) is preferably about 0.5-2 ml/h, in particular about 0.7 ml/h.

17. The method according to claim 10, characterised in that the material jet (8) comprises a polymer-solvent mixture, in particular
a solution of 5 wt.-% of polydioxanone (PDS) in hexafluoro2propanol, or
a solution of 5 wt.-% of thermoplastic polyurethane (TPU) in hexafluoro2propanol.
